(19) United States
(12) Patent Application Publication
(10) Pub. No.: US 2004/0230151 A1
(43) Pub. Date: Nov. 18, 2004

(54) Foot protective structure for shoe sole

(76) Inventor: Pei-Lin Chen, Taichung Hsien (TW)

Correspondence Address: CHARLES E. BAXLEY, ESQ., 90 JOHN STREET, THIRD FLOOR, NEW YORK, NY, 10038, US

(21) Appl. No.: 10/843,546

(22) Filed: May 10, 2004

(30) Foreign Application Priority Data
May 14, 2003 (CN) 03257585.8

(51) Int. Cl. A61F 13/00

(52) U.S. Cl. 602/61

(57) ABSTRACT

A foot protective structure includes a main body having a plurality of movable plates, and a coating layer enclosed around the movable plates. Thus, the movable plates are movable relative to the main body and are movable relative to each other so as to fit movement of the shoe sole, so that the foot protective structure will not limit movement of the user's foot, thereby causing a comfortable sensation to the user. In addition, the coating layer covers the movable plates to prevent the melted material of the shoe sole from entering the gaps between the movable plates, so that the movable plates are separated from each other and will not be bonded with each other after the plastic injection process, thereby enhancing the productivity of the foot protective structure.

### BACKGROUND OF THE INVENTION

1. Field of the Invention

The present invention relates to a foot protective structure, and more particularly to a foot protective structure for a shoe sole.

2. Description of the Related Art

A first conventional foot protective structure 1A for a shoe sole in accordance with the prior art shown in FIGS. 10 and 11 comprises a plurality of fiber layers 1 laminated with each other. The conventional foot protective structure 1A is mounted in a shoe sole (not shown). In such a manner, each of the fiber layers 1 includes a plurality of fiber lines 102, 104, 106 and 108 interwoven in a staggered manner, so that the conventional foot protective structure 1A has an enhanced strength to prevent a sharp object, such as a nail or the like, from penetrating the shoe sole so as to protect a user's foot. However, as shown in FIG. 11, the conventional foot protective structure 1A has a fixed size, so that the front edge of the conventional foot protective structure 1A cannot be bent and easily limits movement of the user's foot when moving, thereby easily causing an uncomfortable sensation to the user.

A second conventional foot protective structure 2A for a shoe sole in accordance with the prior art shown in FIG. 12 comprises a front part 3 and a rear part 2 juxtaposed to the front part 3. Thus, the rear part 2 is movable relative to the front part 3 to satisfy the ergonomic design and will not limit movement of the user's foot, thereby causing a comfortable sensation to the user. However, a gap is easily formed between the front part 3 and the rear part 2 during a long-term utilization, thereby decreasing the protective effect of the second conventional foot protective structure 2A.

### SUMMARY OF THE INVENTION

The primary objective of the present invention is to provide a foot protective structure for a shoe sole.

Another objective of the present invention is to provide a foot protective structure, wherein the movable plates are movable relative to the main body and are movable relative to each other so as to fit movement of the shoe sole, so that the foot protective structure satisfies the ergonomic design and will not limit movement of the user's foot, thereby causing a comfortable sensation to the user.

A further objective of the present invention is to provide a foot protective structure, wherein when the foot protective structure and the shoe sole are placed into a die to be worked during the plastic injection process, the coating layer covers the movable plates completely to prevent the melted material of the shoe sole from entering the gaps between the movable plates, so that the movable plates are separated from each other and will not be bonded with each other after the plastic injection process, thereby enhancing the productivity of the foot protective structure.

A further objective of the present invention is to provide a foot protective structure that is manufactured by laminating the movable plates and then enclosing the coating layer around the movable plates, so that the foot protective structure is made easily and conveniently, thereby decreasing costs of fabrication.

In accordance with the present invention, there is provided a foot protective structure, comprising:

a main body having a plurality of movable plates.

In addition, the foot protective structure further comprises a coating layer enclosed around the movable plates completely.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a foot protective structure in accordance with the preferred embodiment of the present invention;

FIG. 2 is a side plan cross-sectional view of the foot protective structure as shown in FIG. 1;

FIG. 3 is a schematic operational view of the foot protective structure as shown in FIG. 2 in use;

FIG. 4 is a side plan cross-sectional view showing making of the foot protective structure as shown in FIG. 1;

FIG. 5 is a partially cut-away side plan view of the foot protective structure as shown in FIG. 1;

FIG. 6 is a partially perspective cross-sectional view of a foot protective structure in accordance with another embodiment of the present invention;

FIG. 7 is a perspective view of the foot protective structure as shown in FIG. 6;

FIG. 8 is a side plan cross-sectional view of the foot protective structure as shown in FIG. 7;

FIG. 9 is a schematic operational view of the foot protective structure as shown in FIG. 8 in use;

FIG. 10 is a plan exploded view of a first conventional foot protective structure in accordance with the prior art;

FIG. 11 is a perspective view of the first conventional foot protective structure in accordance with the prior art; and

FIG. 12 is an exploded perspective view of a second conventional foot protective structure in accordance with the prior art.

### DETAILED DESCRIPTION OF THE INVENTION

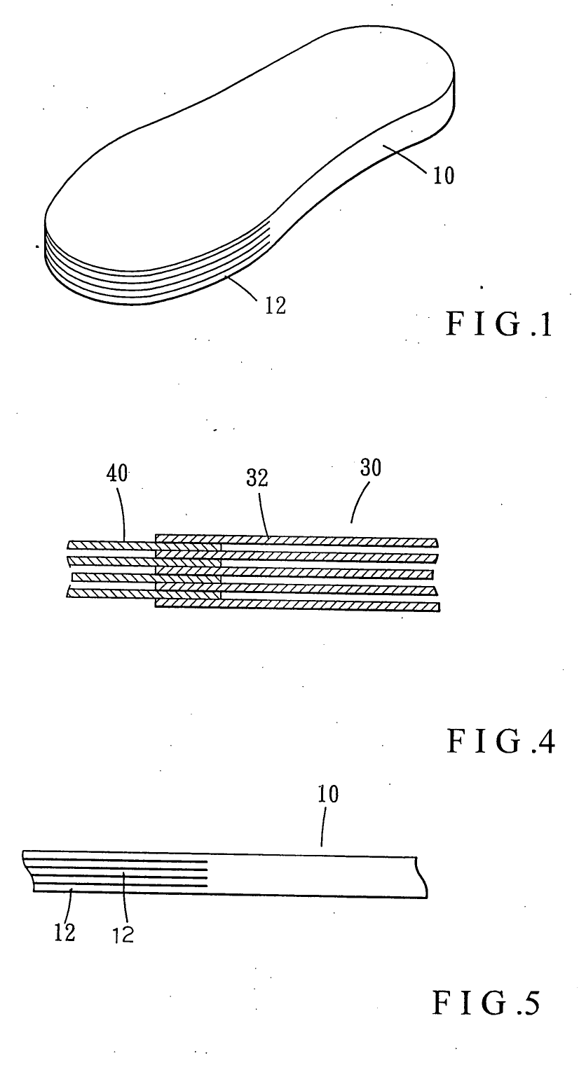

Referring to the drawings and initially to FIG. 1, a foot protective structure in accordance with the preferred embodiment of the present invention comprises a main body 10 having a plurality of movable plates 12. Preferably, the movable plates 12 are integrally formed on an end of the main body 10. The movable plates 12 laminate each other and are separated from each other, so that the movable plates 12 are movable relative to the main body 10. In addition, the movable plates 12 are movable relative to each other. Preferably, each of the movable plates 12 is made of glass fibers interwoven in a staggered manner. Alternatively, each of the movable plates 12 is made of carbon fibers interwoven in a staggered manner. Alternatively, each of the movable plates 12 is made of composite fibers interwoven in a staggered manner. Alternatively, each of the movable plates 12 is made of a plastic sheet.

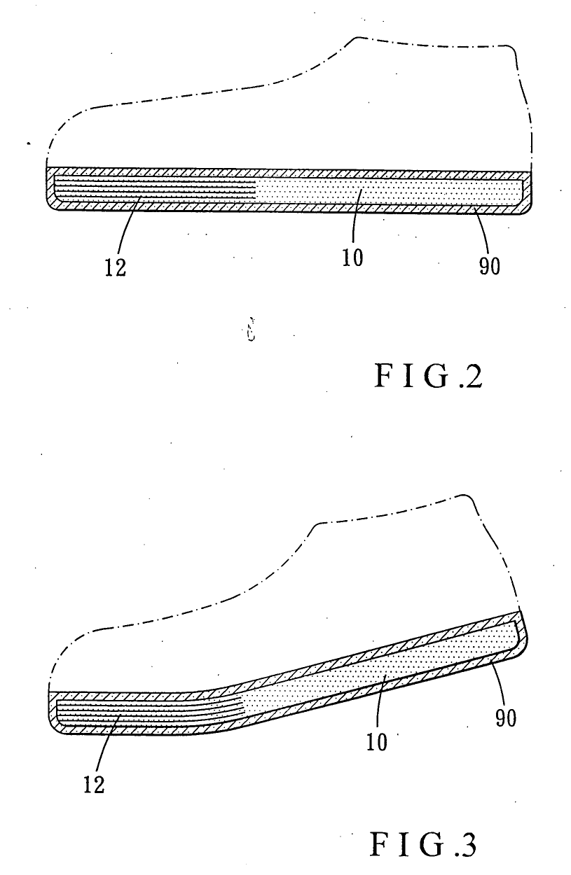

As shown in FIGS. 2 and 3, the foot protective structure is mounted in a shoe sole 90, with the main body 10 and the movable plates 12 being hidden in the shoe sole 90.

In such a manner, the foot protective structure has an enhanced strength to prevent a sharp object, such as a nail or the like, from penetrating the shoe sole 90 so as to protect a user's foot. In addition, the movable plates 12 are movable relative to the main body 10 and are movable relative to each other so as to fit movement of the shoe sole 90, so that the foot protective structure satisfies the ergonomic design and will not limit movement of the user's foot, thereby causing a comfortable sensation to the user. Further, the movable plates 12 are separated from each other, so that the thickness of the movable plates 12 is reduced, thereby decreasing costs of fabrication.

As shown in FIGS. 4 and 5, a method for making the foot protective structure in accordance with the preferred embodiment of the present invention comprises the steps of: laminating a plurality of fiber materials 32 to form a blank plate 30, separating the front ends of the fiber materials 32 by a plurality of spacers 40, placing the blank plate 30 and the spacers 40 into a die to perform a heat pressing process to solidify the fiber materials 32 so that the rear ends of the fiber materials 32 are solidified and combined to form the main body 10 and the front ends of the fiber materials 32 are solidified to form the movable plates 12 which are separated from each other, and finally removing the spacers 40 from the blank plate 30, thereby forming a foot protective structure comprising the main body 10 and the movable plates 12.

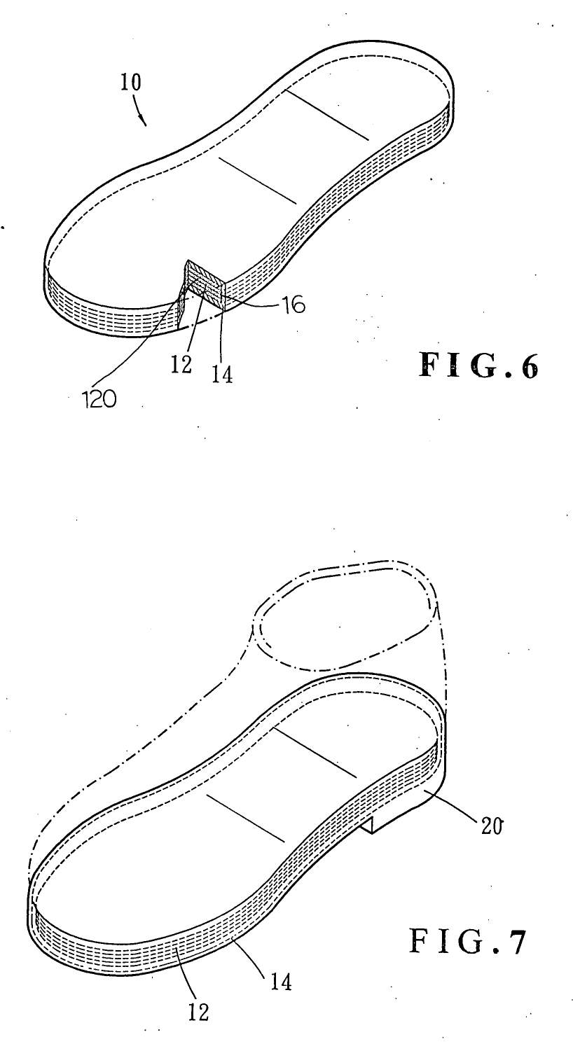

Referring to FIG. 6, the movable plates 12 are laminated with each other in a vertical manner to form the main body 10. Alternatively, the movable plates 12 have first ends combined with each other and integrally connected to the main body 10 and second ends separated from each other. In addition, the foot protective structure further comprises a coating layer 14 enclosed around the movable plates 12. Preferably, the coating layer 14 is made of a cloth closed by stitching. Alternatively, the coating layer 14 is formed by a plastic material worked by high-frequency waves. A gap 16 is formed between the movable plates 12 and the coating layer 14, and a gap 120 is formed between any two adjacent movable plates 12, so that the movable plates 12 are moved freely.

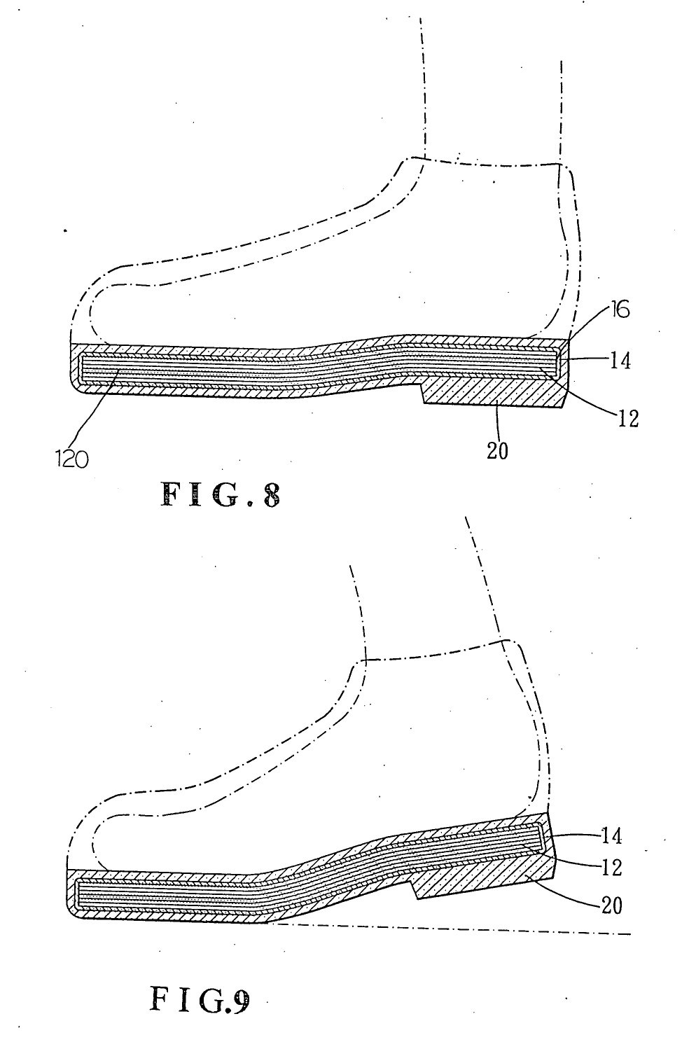

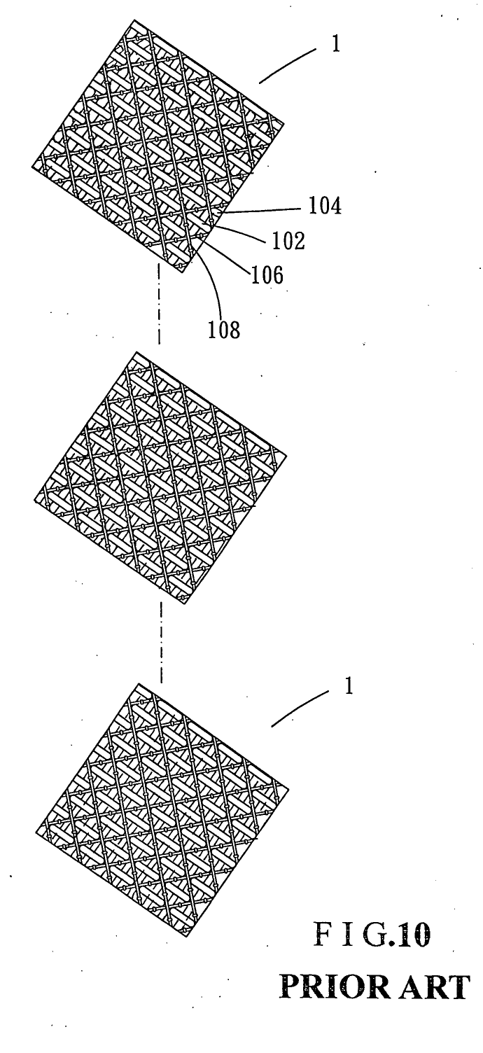

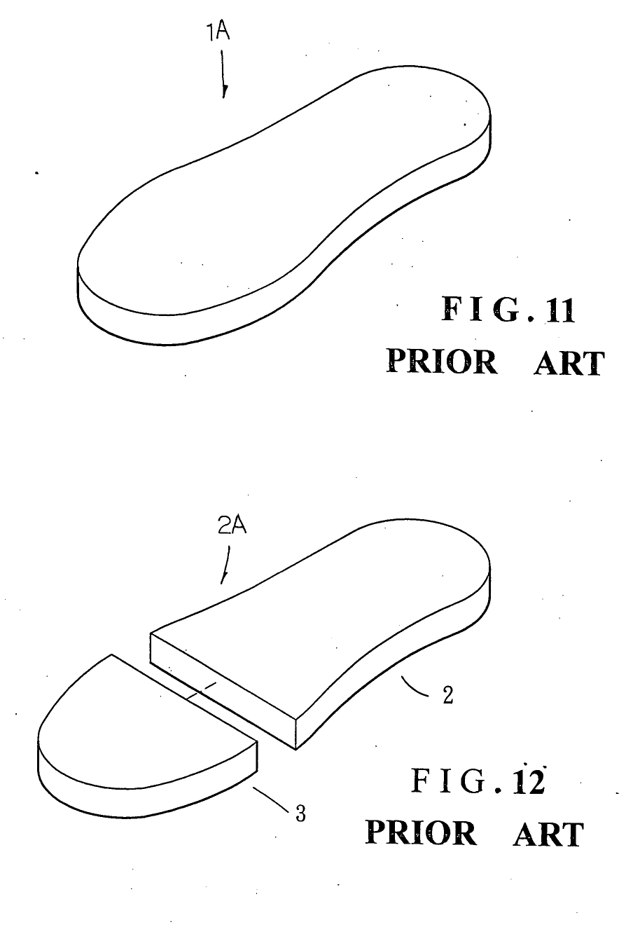

As shown in FIGS. 7-9, the foot protective structure is mounted in a shoe sole 20, with the movable plates 12 and the coating layer 14 being hidden in the shoe sole 20.

In such a manner, when the foot protective structure and the shoe sole 20 are placed into a die to be worked during the plastic injection process, the coating layer 14 covers the movable plates 12 completely to prevent the melted material of the shoe sole 20 from entering the gaps 120 between the movable plates 12, so that the movable plates 12 are separated from each other completely and will not be bonded with each other after the plastic injection process, thereby enhancing the productivity of the foot protective structure. In addition, the foot protective structure is made by laminating the movable plates 12 and enclosing the coating layer 14 around the movable plates 12, so that the foot protective structure is made easily and conveniently, thereby decreasing costs of fabrication.

Although the invention has been explained in relation to its preferred embodiment(s) as mentioned above, it is to be understood that many other possible modifications and variations can be made without departing from the scope of the present invention. It is, therefore, contemplated that the appended claim or claims will cover such modifications and variations that fall within the true scope of the invention.

What is claimed is:

1. A foot protective structure, comprising:
a main body having a plurality of movable plates.

2. The foot protective structure in accordance with claim 1, wherein the movable plates are integrally formed on an end of the main body.

3. The foot protective structure in accordance with claim 1, wherein the movable plates are separated from each other.

4. The foot protective structure in accordance with claim 1, wherein the movable plates are movable relative to the main body.

5. The foot protective structure in accordance with claim 1, wherein the movable plates are movable relative to each other.

6. The foot protective structure in accordance with claim 1, wherein each of the movable plates is made of glass fibers interwoven in a staggered manner.

7. The foot protective structure in accordance with claim 1, wherein each of the movable plates is made of carbon fibers interwoven in a staggered manner.

8. The foot protective structure in accordance with claim 1, wherein each of the movable plates is made of composite fibers interwoven in a staggered manner.

9. The foot protective structure in accordance with claim 1, wherein each of the movable plates is made of a plastic sheet.

10. The foot protective structure in accordance with claim 1, wherein the movable plates are laminated with each other in a vertical manner to form the main body.

11. The foot protective structure in accordance with claim 1, wherein the movable plates have first ends combined with each other and integrally connected to the main body and second ends separated from each other.

12. The foot protective structure in accordance with claim 1, further comprising a coating layer enclosed around the movable plates completely.

13. The foot protective structure in accordance with claim 12, wherein the coating layer is made of a cloth closed by stitching.

14. The foot protective structure in accordance with claim 12, wherein the coating layer is formed by a plastic material worked by high-frequency waves.

15. The foot protective structure in accordance with claim 12, wherein a gap is formed between the movable plates and the coating layer.

16. The foot protective structure in accordance with claim 1, wherein a gap is formed between any two adjacent movable plates, so that the movable plates are movable freely.
